(19) United States
(12) Patent Application Publication
(10) Pub. No.: US 2008/0132531 A1
(43) Pub. Date: Jun. 5, 2008

(54) SYNERGISTIC COMBINATIONS OF NORKETAMINE AND OPIOID ANALGESICS

(75) Inventors: Joseph R. Holtman, Lexington, KY (US); Peter A. Crooks, Nicholasville, KY (US)

Correspondence Address: FOLEY AND LARDNER LLP;SUITE 500, 3000 K STREET NW, WASHINGTON, DC, 20007, US

(73) Assignee: Yaupon Therapeutics, Inc.

(21) Appl. No.: 11/558,782

(22) Filed: Nov. 10, 2006

Related U.S. Application Data
(60) Provisional application No. 60/735,921, filed on Nov. 14, 2005.

(30) Foreign Application Priority Data
Nov. 11, 2005 (GB) 0523031.3

(51) Int. Cl. A61K 31/439; A61P 29/00; A61P 25/00; A61K 31/135; A61K 31/445; A61K 31/4355

(52) U.S. Cl. 514/282; 514/646; 514/329; 514/326; 514/281; 514/183

(57) ABSTRACT

The present invention relates to methods of alleviating pain with the administration of norketamine with a narcotic. More particularly, the invention provides a method of alleviating pain through the administration of a dose of norketamine, which, if administered alone would provide sub-optimal analgesic relief, yet provides analgesic relief when combined with a narcotic. In some embodiments, the combination of norketamine with a narcotic, further allows for the administration of a dose narcotic, which would be sub-optimal if used alone, but provides adequate pain relief in combination with norketamine. The invention relates to self-management of pain on an outpatient basis comprising administering via conventional routes, including transdermal, nasal, rectal, oral, transmucosal, intravenous, intramuscular, and other routes, one or more doses of norketamine/opioid compositions effective to alleviate pain to a subject suffering from pain. Uses of norketamine/opioid compositions would also apply, to treating headaches, drug abuse, mood and anxiety disorders, as well as other, neuropsychiatric disorders, both motoric and cognitive, such as Alzheimer's disease, Parkinson's syndrome, which are thought to be caused by neurodegeneration.

### CROSS-REFERENCE TO RELATED PATENT APPLICATIONS

This application claims priority from United Kingdom Application 0523031.3, filed Nov. 11, 2005 and U.S. Provisional Application 60/735,921, filed Nov. 14, 2005, both of which are incorporated by reference herein in their entireties.

### TECHNICAL FIELD

The present invention generally relates to analgesic drugs and methods of use their use. More particularly, the invention relates to pharmaceuticals comprising a combination of norketamine and a narcotic and methods of their use for the management of chronic pain.

### BACKGROUND

Norketamine (2-(2-chlorophenyl)-2-amino-cyclohexanone) is one of the principal metabolic products of ketamine (2-(2-chlorophenyl)-2-(methylamino)-cyclohexanone), which is a general anesthetic used by anesthesiologists, veterinarians, and researchers. Current pharmaceutical compositions of ketamine are racemic mixtures of S- and R-ketamine, though S-ketamine has been found recently to be twice as potent as R-ketamine and to allow faster recovery with fewer negative side effects than the racemic mixture (C. S. T. Aun, 1999, Br. J. Anaesthesia 83: 29-41). Studies have shown that ketamine is converted metabolically through demethylation to norketamine, in vivo, at rates dependent on the route of administration, with oral and rectal administrations having the fastest rates due to a high degree of first pass metabolism in the liver (see, e.g., Grant et al., 1981, Br. J. Anaesth. 53: 805-810; Grant et al., 1981, Br. J. Anaesth. 55: 1107-1111; Leung et al., 1985, J. Med. Chem. 29: 2396-2399; Malinovsky et al., 1996, Br. J. Anaesthesia 77: 203-207). Norketamine binds the NMDA receptor less tightly than either S- or R-ketamine (Ebert et al., 1997, Eur. J. Pharm. 333: 99-104) and norketamine is speculated to have an anesthetic and analgesic potency one third that of ketamine (C. S. T. Aun, 1999, Br. J. Anaesthesia 83: 29-41), perhaps explaining the absence of administration of norketamine as an analgesic in the art.

Ketamine also has analgesic properties (Domino et al., 1965, Clin. Pharmacol. Ther. 6:279); profound analgesia can be achieved with subanesthetic doses of ketamine (Bovill, 1971, Br. J. Anaesth. 43:496; Sadove et al., 1971, Anesth. Analg. 50:452-457). The drug is administered by various routes, including i.v., i.m., caudal, intrathecal, oral, rectal, and subcutaneous (s.c.) (see, e.g., Oshima et al., 1990, Can. J. Anaesth. 37:385-386).

Management of pain, and particularly chronic pain, is complex and frequently unsuccessful. The first line of treatment usually involves administration of opioid agonists, e.g., narcotics such as morphine (see, e.g., Anderson and Brill, 1992, Semin. Anesth. 11: 158-171). However, rapid tolerance and marked resistance to narcotics frequently develop, thus rendering these agents ineffective (see, e.g., Abram, 1993, Reg. Anesth. 18(SUPPL):406-413). Non-competitive N-methyl-D-aspartate (NMDA) receptor antagonists, such as ketamine and norketamine, have been reported to interfere with the development of tolerance to the analgesic effects of morphine, possibly through blockade of the NMDA receptor rather than from “side-effects” of the antagonist, since the antagonists were not found to reverse tolerance (Trujillo and Akil, 1994, Brain Res. 633:178-188).

Often, pain management involves administration of a plethora of drugs, such as narcotics, agonist-antagonist agents, butorphanols, benzodiazepines, GABA stimulators, barbiturates, barbiturate-like drugs, orally, e.g., in a pill or liquid formulation, or by i.v. or i.m. injection. Opioid agonists and antagonists may be combined. Thus, a combination of drugs can have offsetting or compounding effects. More problematic is the possibility of adverse side effects, particularly gastric distress that accompanies oral administration, or the fear that injections can inspire.

U.S. Pat. Nos. 5,543,434 and 6,248,789 B1 disclose transmucosal and nasal administrations of ketamine for the management of pain and to reduce drug dependency. Under the methods of Weg, dosages must be kept low in order to avoid the dysphoric side effects attributable to ketamine. However, studies have indicated that norketamine, delivered intravenously (Leung et al., 1985, J. Med. Chem. 29: 2396-2399) or intraspinally (Shimoyama et al., 1999, Pain 81: 85-93) to rats, produced fewer of the adverse sequelae than an equal dose of ketamine.

Thus, there is a need for pain management therapies, which reduce the dose of analgesics, including the narcotics. This and other needs in the art have been addressed by the instant invention, which is based on a novel finding that S-norketamine, R-norketamine, racemic mixtures thereof, and prodrugs thereof can be used to alleviate pain safely and effectively in doses that would have been sub-optimal or ineffective alone, but provide analgesic relief when in combination with a narcotic. The invention also provides a method of alleviating pain with administration of norketamine and a narcotic in doses that would have been sub-optimal or ineffective if administered alone, but provide analgesic relief when in combination.

The citation or identification of any reference in this application shall not be construed as an admission that such reference is available as prior art to the present invention. However, all references and citations identified in this application are incorporated in their entirety by reference in the present application.

### SUMMARY OF THE INVENTION

The object of the present invention is to provide a drug composition comprising racemic norketamine, (S)-norketamine, (R)-norketamine, their respective salts, solvates, or prodrugs, or any combinations thereof in combination with an opioid, provided that the effective amount of the norketamine, if administered in the absence of the opioid, would be insufficient to exert an optimal analgesic effect on the subject. Prodrugs of norketamine may be provided through the chemical linking of norketamine to a variety of carboxylic acids and other substituents to afford the formulae shown in Structures 1 and 2 below.

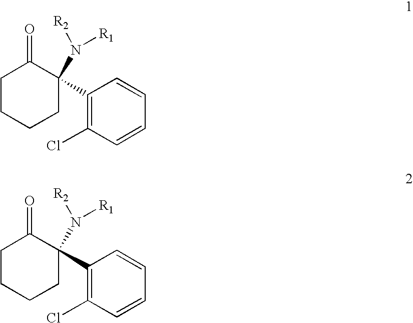

and wherein R₃ and R₄ are phenyl, aryl, azaaryl, alkyl, branched alkyl, cycloalkyl, alkenyl, cycloalkenyl; where R₅═OH or SH;
and where R₆=alkyl, branched alkyl;
racemic mixtures of compounds of formula 1 and formula 2 in which R₁═H and R₂ can be any of the groups recited above, including H; and pharmaceutically acceptable salts and solvates thereof.

When R-norketamine is in the free base form, it has a (+) optical rotation and when in the salt form a (−) optical rotation. S-norketamine has a (−) optical rotation when in the free base form and when in the salt form a (+) optical rotation.

As well, the invention provides a method of pain treatment where the effective amount of the opioid, if administered in the absence of a norketamine compound, would be insufficient to exert its optimum analgesic effect on the subject. The norketamine compound and the opioid ingredients may be administered separately or concomitantly and synergistically contribute to achieve an optimum analgesic effect.

Examples of opioids include, but are not limited to fentanyl, sefentanil, alfentanil, morphine, hydromorphine, oxymorphine, methadone, oxycodone, hydrocodone, remifentanil, dihydrocodeine, ethylmorphine, nalbuphine, buprenorphine, dihydromorphine, normorphine, dihydroetorphine, butorphanol, pentazocine, phenazocine, codeine, meperidine, propoxyphene, tramadol, levorphanol, L-acetylmethadol, diacetylmorphine (heroin), etorphine, normethadone, noroxycodone, and norlevorphanol. In one preferred embodiment, the opioid is morphine. Opioids are understand by one of skill in the art to include their salt forms.

In another embodiment of the present invention, a method of inhibiting tolerance to a narcotic analgesic in a subject in need thereof is provided, comprising co-administering to a subject in need thereof (S)-norketamine, (R)-norketamine, their respective salts, solvates, or prodrugs, or any combinations thereof with a narcotic analgesic, in which the narcotic analgesic, if administered in the absence of the (S)-norketamine, (R)-norketamine, their respective salts, solvates, or prodrugs, or any combinations thereof, would induce in the subject a tolerance for the narcotic analgesic. The invention may also be effective where the narcotic analgesic could induce in the subject a tolerance for the narcotic analgesic after about one week of daily administration.

Compositions of the present invention may be delivered by any of a number of routes, including transdermal, nasal, rectal, vaginal, oral, transmucosal, intravenous, intramuscular, caudal, intrathecal, and subcutaneous. In a further embodiment, the present invention provides for pulmonary administration by inhalation. Transdermal, nasal, and pulmonary administration advantageously allows for patient self administration of the drug, which provides for pain management on an outpatient basis. Moreover, administration in transdermal patches, nasal sprays, and inhalers are generally socially acceptable.

In yet another embodiment of the invention, a device is provided for patient self-administration of norketamine/opioid compositions. The device of the invention may comprise a pulmonary inhaler containing a formulation of norketamine/opioid compositions, optionally with a pharmaceutically acceptable dispersant, wherein the device is metered to disperse an amount of the formulation that contains a dose of norketamine with narcotic effective to alleviate pain. The dispersant may be a surfactant, such as, but not limited to, polyoxyethylene fatty acid esters, polyoxyethylene fatty acid alcohols, and polyeoxyethylene sorbitan fatty acid esters.

In one specific embodiment, the formulation is a dry powder formulation in which the norketamine/narcotic composition is present as a finely divided powder. The dry powder formulation can further comprise a bulking agent, such as, but not limited to, lactose, sorbitol, sucrose and mannitol, or the norketamine/opioid compositions may be associated with carrier particles.

In another specific embodiment, the formulation is a liquid formulation, optionally comprising a pharmaceutically acceptable diluent, such as, but not limited to, sterile water, saline, buffered saline and dextrose solution.

In further embodiments, the formulation further comprises a benzodiazepine in a concentration such that the metered amount of the formulation dispersed by the device contains a dose of the benzodiazepine effective to inhibit dysphoria, or a narcotic in a concentration such that the metered amount of the formulation dispersed by the device contains a dose of the narcotic effective to alleviate pain.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 shows norketamine in dose-dependent antinociception in a rodent model of neuropathy (mechanical test).

FIG. 2 shows norketamine in dose-dependent antinociception in a rodent model of neuropathy (thermal test).

FIG. 3 shows antinocipentive efficacy of norketamine vs. ketamine.

FIG. 4 shows antinocipentive efficacy of S-norketamine.

FIG. 5 shows antinocipentive efficacy of R-norketamine.

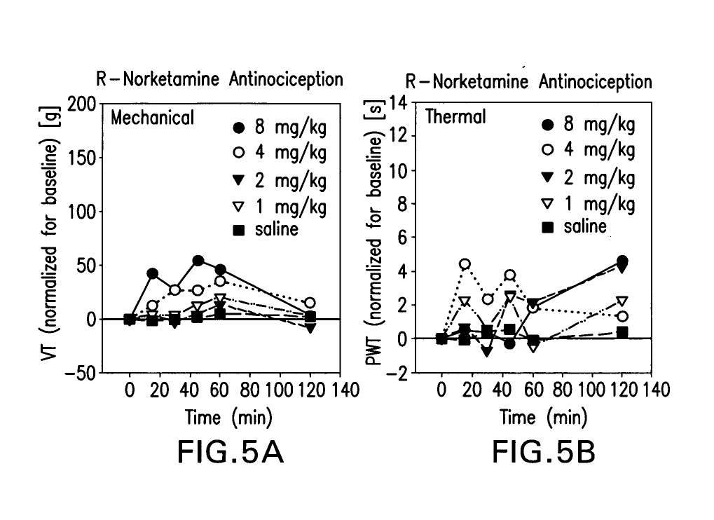

FIG. 6 compares antinocipentive efficacy of S- and R-norketamine.

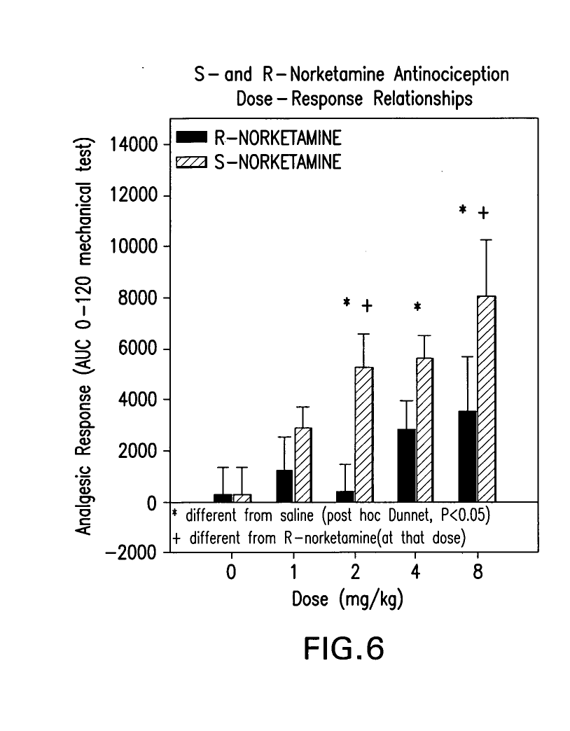

FIG. 7 shows the correlation between antinocipentive efficacy of norketamine vs. its plasma levels.

FIG. 8 shows the affect of norketamine on motor function.

FIG. 9 shows the affect of norketamine to induce ataxia.

FIG. 10 shows the synergistic analgesic effect of morphine with racemic norketamine following IP administration.

FIG. 11 shows the synergistic analgesic effect of morphine with S-norketamine following IP administration. From left to right the bars represent S-norketamine, 3 mg/kg, morphine 3 mg/kg, and S-norketamine, 3 mg/kg with morphine 3 mg/kg.

FIG. 12 shows the synergistic analgesic effect of morphine with S-norketamine following intrathecal administration. From left to right the bars represent S-norketamine 100 mcg (n=7), morphine 0.5 mcg (n=4) and S-norketamine 100 mcg with morphine 0.5 mcg (n=6).

FIG. 13 shows the reduction of morphine tolerance with S-norketamine administration.

FIG. 14 shows the analgesic effect of morphine after IP administration.

FIG. 15 shows the analgesic effect of morphine after IT administration.

FIG. 16 shows the effect of S-norketamine alone after IP and IT administration.

FIG. 17 shows the synergistic effect of morphine by S-norketamine after IP administration.

FIG. 18 shows the synergistic effect of morphine by S-norketamine after IT administration.

FIG. 19 also shows the synergistic effect of S-norketamine and oxycodone.

FIG. 20 shows that tolerance of oxycodone is inhibited by S-norketamine.

FIG. 21 shows the synergistic effect of S-norketamine and morphine

### DETAILED DESCRIPTION OF THE INVENTION

The present invention relates to the administration of norketamine and a narcotic in combination for the treatment of pain. More specifically, the present invention provides administration of sub-analgesic doses of norketamine and/or the narcotic, which, when used in combination, provides an analgesic effect. The invention also provides a method and device for patient self administration of the described drugs for pain management.

The present invention contemplates the use of racemic or enantiomericaly pure compositions of norketamine. S- and R-norketamine are described by formulae 1 and 2 [below], respectively, wherein R₁ and R₂ are hydrogen. While the invention will be

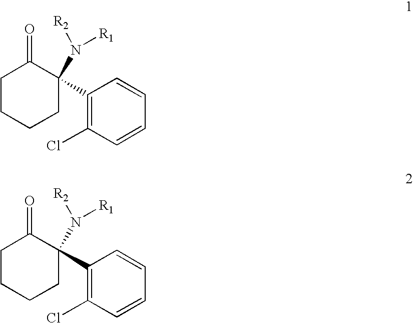

described, in significant part, with reference to “norketamine,” analgesic compositions described herein may also comprise prodrugs (i.e., derivatives) of norketamine as described in detail in U.S. Patent Application Publication No. 20040248964, filed on Nov. 18, 2003, the disclosure of which is incorporated herein in its entirety by reference. Thus, unless more specific language is recited, the term “norketamine” is used herein to encompass the individual isomers of norketamine and derivatives thereof.

In specific embodiments, norketamine refers to salts of norketamine, such as norketamine hydrochloride. There is no limitation on the nature of these salts, provided that, when used for therapeutic purposes, they are pharmaceutically acceptable, which, as is well-known in the art, means that they do not have reduced activity or increased toxicity compared with the free compounds. Examples of these salts include: salts with an inorganic acid such as hydrochloric acid, hydrobromic acid, hydriodic acid, nitric acid, perchloric acid, sulfuric acid or phosphoric acid; and salts with an organic acid, such as methanesulfonic acid, trifluoromethanesulfonic acid, ethanesulfonic acid, benzenesulfonic acid, p-toluenesulfonic acid, fumaric acid, oxalic acid, maleic acid, citric acid, succinic acid, tartaric acid; and other mineral and carboxylic acids well known to those skilled in the art. Examples of salts with inorganic cations such as sodium, potassium, calcium, magnesium, lithium, aluminum, zinc, etc; and salts formed with pharmaceutically acceptable amines such as ammonia, alkylamines, hydroxyalkylamines, lysine, arginine, N-methylglucamine, procaine and the like.

“Prodrugs of norketamine” is used herein to refer to all compounds that may be converted physiologically to norketamine. While it is well known that ketamine is metabolized to norketamine in vivo, it is important to note that ketamine is not to be considered a prodrug of norketamine, and the term “norketamine prodrug” in all its forms specifically excludes ketamine as used in this application.

Prodrugs of norketamine may be provided through the chemical linking of norketamine to a variety of carboxylic acids and other substituents to afford the formulae shown in Structures 1 and 2 below.

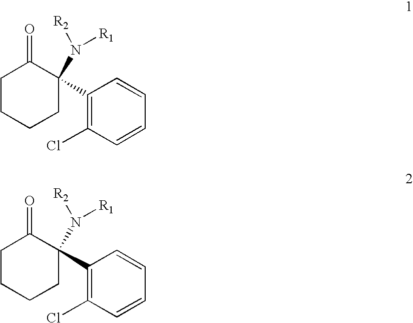

wherein:

### R₁=Methyl, R₂═CH₂OCOR₃

### R₁═H, R₂═CH₂OCOR₃

### R₁=Methyl, R₂═CH₂COOR₃

### R₁═H, R₂═CH₂COOR₃

### R₁=Methyl, R₂═COOR₃

### R₁═H, R₂═COOR₃

### R₁=Methyl, R₂═COOCH₂CH₂N(CH₃)₂

### R₁═H, R₂═COOCH₂CH₂N(CH₃)₂

### R₁=Methyl, R₂═COOCH(R₃)OCOR₄

### R₁═H, R₂═COOCH(R₃)OCOR₄

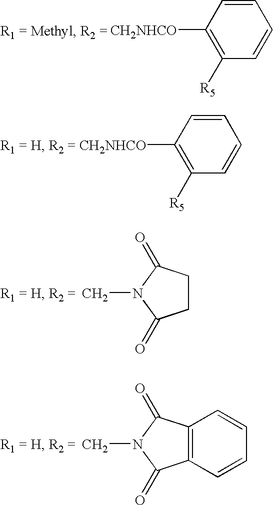

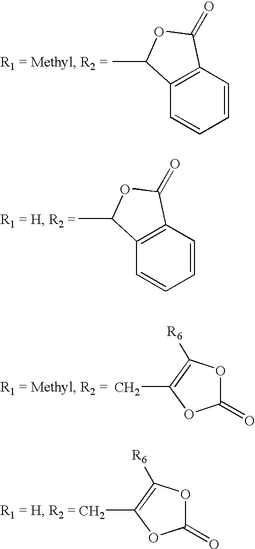

and wherein R₃ and R₄ are phenyl, aryl, azaaryl, alkyl, branched alkyl, cycloalkyl, alkenyl, cycloalkenyl; where R₅═OH or SH;
and where R₆=alkyl, branched alkyl;
racemic mixtures of compounds of formula 1 and formula 2 in which R₁═H and R₂ can be any of the groups recited above, including H; and pharmaceutically acceptable salts and solvates thereof. Aryl, azaaryl, alkyl, branched alkyl, cycloalkyl, alkenyl, cycloalkenyl moieties can be C₁-C₆.

“Narcotics” are defined herein as opioids and interchangeably used. Narcotics and opiods are ligands that bind to the mu, delta and kappa receptors. Narcotics suitable in the present invention, include, but are not limited to, fentanyl, sefentanil, alfentanil, morphine, hydromorphine, oxymorphine, methadone, oxycodone, hydrocodone, remifentanil, dihydrocodeine, ethylmorphine, nalbuphine, buprenorphine, dihydromorphine, normorphine, dihydroetorphine, butorphanol, pentazocine, phenazocine, codeine, meperidine, propoxyphene, tramadol, levorphanol, L-acetylmethadol, diacetylmorphine (heroin), etorphine, normethadone, noroxycodone, and norlevorphanol. Morphine is a preferred narcotic in some embodiments of the invention. Narcotics of the present invention can be in salt form. Also, narcotics of the present invention can be in prodrug form. Exemplary prodrugs include the prodrug forms described above for norketamine.

An “optimal” dose is defined as a dose of an analgesic, when taken alone, is sufficient to provide analgesic relief. In the rat, for example, an optimal dose of norketamine is about 8 mg/kg intraperitoneally (IP). A “sub-optimal” dose is defined as about 1 to about 60% of the optimal dose used to induce analgesia; more preferably about 5% to about 40%, and even more preferably about 10% to about 20%. A “sub-analgesic” does is defined as a dose at which little to no analgesic effect is provided. For example, a sub-analgesic dose of norketamine is less than about 3 mg/kg. Typically, a sub-analgesic dose correlates with less than about 5 AUC units or less than about 5% MPE (maximum possible effect).

The actual dose will vary, of course, depending on the body weight of the patient, the severity of the pain, the route of administration, such as oral verses a parenteral route, the nature of medications administered concurrently, the number of doses to be administered per day, and other factors generally considered by the ordinary skilled physician in the administration of drugs. Exemplary dosage ranges are 0.05 to 500 mg/kg, more preferably 0.5 to 50 mg kg. Exemplary ratios of opioid to norketamine or 0.05 to 50:1, more preferably 0.1 to 10:1.

As well, the apparent dose for analgesia will often depend on the test model used. Protocols for determining optimal analgesic doses of a given drug in pain management in animal models are known in the art.

One of these protocols will now be described with respect to the invention.

### Tail-Flick Test

A dose response curve was generated by determining the analgesic effects of combining a constant dose of an opioid (e.g., morphine) along with an increasing dose of R,S-norketamine, Male and female Sprague-Dawley rats (n=8/sex) all with an approximate age of 85 to 90 days. Each rat should be weighed, prior to being subjected to any tests, on the day of the experiment. Experiments were performed in 72 hour intervals, and prior to the test, the rats were habituated for three days to handling and the tail-flick procedure without heat exposure.
1 The tail-flick apparatus (IITC Model 33, Life Science, Woodland Hills, Calif.) is pre-warmed for at least 30 minutes.
2 The intensity of the lamp is adjusted so that baseline tail-flick latency for the rats is equal to approximately 2.0 seconds. In the present experiment, the intensity was set to 40% as this was determined to be the ideal intensity from the intensity response curve.
3 The tail-flick apparatus is preferably programmed to use a cut off point of 10 seconds to prevent tissue damage to the rats in the case that the tail does not flick.
4 A rat is placed in a mitten and its tail blackened with ink approximately 2 inches in length at 1 inch from the base of the tail.
5 The tail is then placed flatly in the groove of the tail-flick apparatus.
6 Once heat exposure is initiated, the lamp is set to turn off automatically when the tail moves from the heat source.
7 For each rat, a baseline score is determined prior to injection. Tail-flick latency (“TFL”) is measured twice in an approximate 15 minute intervals and an average of the two times determines the baseline.
8 Once the baseline value is determined, TFL is measured, following the injection of drugs, at times 15, 30, 60 and 120 minutes.

### Solutions:

9 Morphine: 3 mg/kg
   injection volume of 0.5 ml/kg: makeup solution of 6 mg/ml saline.
   For 8 rats 24 mg of morphine should be mixed with 4 ml of saline to give the proper amount of drug needed for a 3 mg/kg dose at an injection volume of 0.5 ml/kg.
10 Norketamine: 3 mg/kg, 1-5 mg/kg, 0.75 mg/kg: injection volume of 0.5 ml/kg: make up solution of 6 mg/ml saline: Method for preparing proper amount of drugs to be used for 8 rats.
   3 mg/kg (A). Mix 12 mg R,S-norketamine with 2 ml saline to give a 6 mg/ml solution to be given at an injection volume of 0.5 ml/kg. Dilute original (A) solution to obtain the following concentrations:
   1.5 mg/kg (B): take 1 ml of (A) and dilute with 1 ml of saline
   0.75 mg/kg (C): take 1 ml of (B) and dilute with 1 ml of saline
11 Saline solution (control): (D)

### Drug Administration (I.P.):

### Total volume (mL) injected is equal to body weight (kg). Each animal is given an injection of morphine that is 0.5 ml/kg body weight, and an injection of norketamine or control that is 0.5 ml/kg body weight.

Example: body weight=250 g=0.25 kg=0.25 ml

For each drug to be injected: 0.5 ml/kg×0.250 kg=0.125 ml injected

### Calculations:

Normalize data for baseline value (postinjection value at each time point−average preinjection baseline).
Calculate area under the curve (AUC₀₋₁₂₀ₘᵢₙ) for normalized data.
Calculate maximum area under the curve (AUCₘₐₓ), assuming 10 second response for each time point.
Calculate % MPE=(AUC₀₋₁₂₀ₘᵢₙ)/(AUCₘₐₓ)×100.
Post-injection thresholds are compared to the baseline threshold using paired t-test.
The difference between doses will be analyzed by 2 way RM ANOVA.
The difference between sex will be analyzed by 2 way RM ANOVA.
All data are presented as mean ±SEM of n rats.

### Method of Calculations:

Normalized data (NOR): subtract average baseline from each value NOR=(post-injection TFL)−(pre-injection baseline)
12 Percent maximum effect (MPE):
   % MPE=(post-injection value−pre-injection baseline)/(cut-off−pre-injection baseline)×100%
13 Area under the time action curve (AUC_{0-120 min}) calculated by trapezoidal rule
   Individual AUC_{0-120 min.}=(average value over the time interval)×(time interval)
   Total AUC_{0-120 min.}: sum of individual AUC
   Example: AUC_{0-120 min.}, % MPE=(15-0 min)×[(MPE0+MPE15)×½]+(30−15)×[(MPE30+MPE15)×½]

### Graphs:

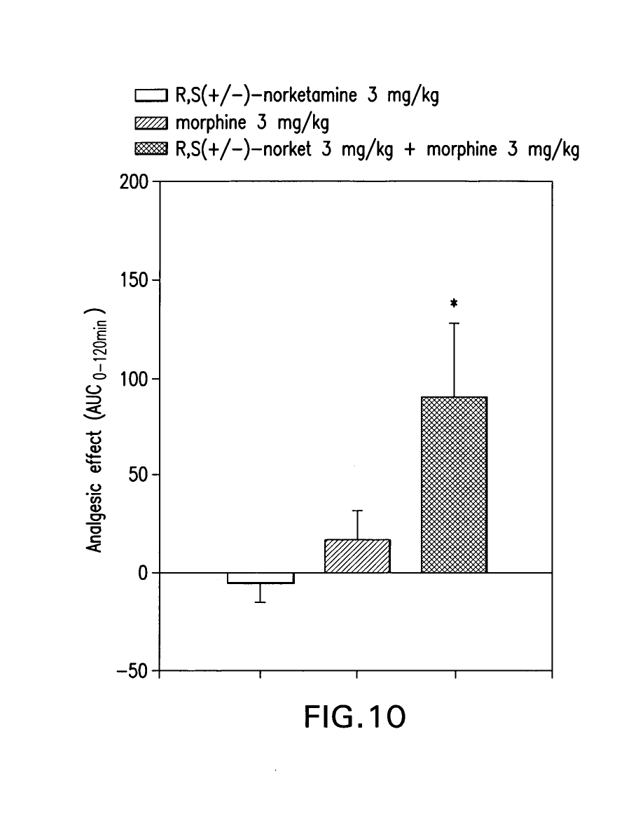

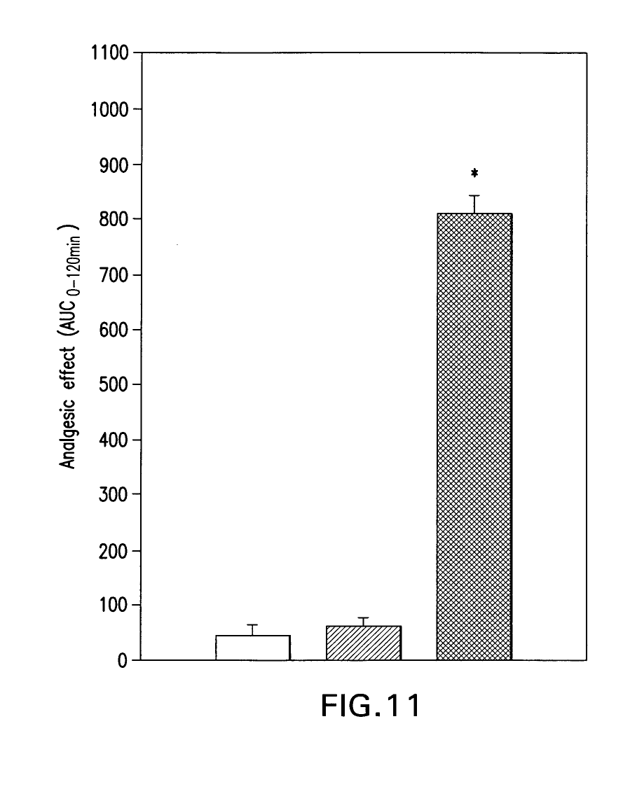

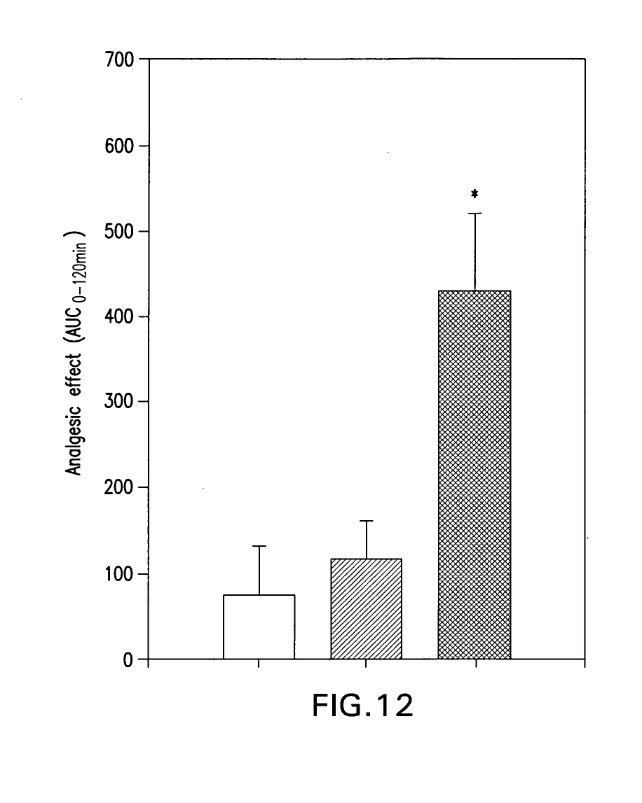

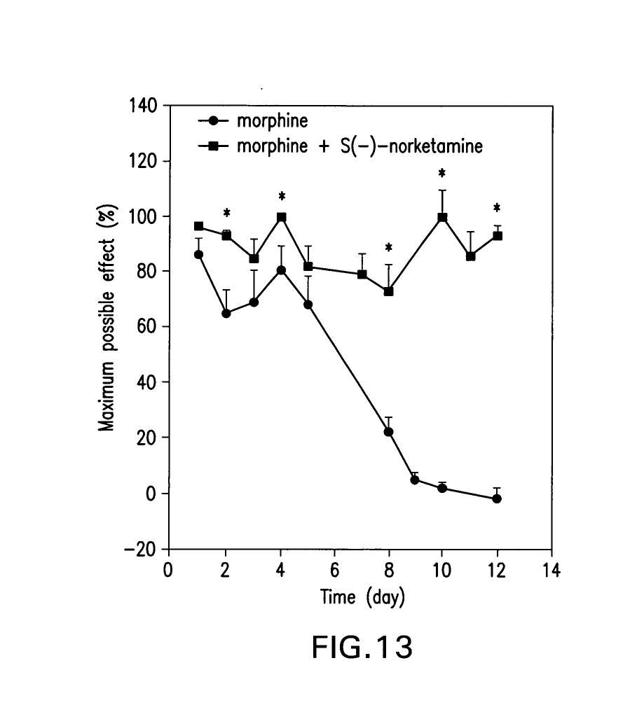

Time action curves were plotted for each dose
Plot data v. concentration for both maximum % MPE and AUC_{0-120 min} were plotted. (FIGS. 10-14).

The invention may be used to alleviate pain from many causes, including but not limited to shock; limb amputation; severe chemical or thermal burn injury; sprains, ligament tears, fractures, wounds and other tissue injuries; dental surgery, procedures and maladies; labor and delivery; during physical therapy; post operative pain; radiation poisoning; cancer; acquired immunodeficiency syndrome (AIDS); epidural (or peridural) fibrosis; failed back surgery and failed laminectomy; sciatica; painful sickle cell crisis; arthritis; autoimmune disease; intractable bladder pain; and the like. Administration of norketamine/narcotic combination is also amenable to hospice use, particularly hospices that specialize in the care of cancer and AIDS patients.

The invention also provides self-management of pain on an outpatient basis comprising administering via conventional routes, including transdermal, nasal, rectal, vaginal, oral, transmucosal, intravenous, intramuscular, intrathecal, epidural, subcutaneous, and other routes, of norketamine with narcotics effective to alleviate pain to a subject suffering from pain. Uses of norketamine/narcotic drugs would also apply, for example, to treating headaches, drug abuse, mood and anxiety disorders, as well as other neuropsychiatric disorders, both motoric and cognitive, such as Alzheimer's disease, Parkinson's syndrome, Restless Leg Syndrome which are thought to be caused by neurodegeneration.

In one embodiment, administration of norketamine with narcotic drugs may relieve or alleviate episodes of acute breakthrough pain or pain related to wind-up that can occur in a chronic pain condition. In a further embodiment, administration of norketamine/narcotic compositions may be used as an adjunct therapy to a conventional treatment regimen for a chronic pain condition to alleviate breakthrough pain or pain related to wind-up.

The norketamine/opioid compositions will preferably be prepared in a formulation or pharmaceutical composition appropriate for administration by the transmucosal route, e.g., nasal, transbuccal, sublingual, vaginal, and rectal; by the oral route (via the gastrointestinal tract, rather than the oral-pharyngeal mucosa); by the pulmonary route (i.e., inhaled); or by the parenteral route, e.g., intravenous, intraarterial, intraperitoneal, intradermal, intramuscular, intraventricular, or subcutaneous. Suitable formulations are discussed in detail, infra. In a further embodiment, the norketamine/narcotic composition can be formulated with a mucosal penetration enhancer to facilitate delivery of the drug. The formulation can also be prepared with pH optimized for solubility, drug stability, absorption through skin or mucosa, and other considerations.

In another embodiment, the dose of norketamine and narcotic, individually, is about 0.01 mg per kg of body weight (0.01 mg/kg) to about 200 mg/kg; preferably about 0.05 mg/kg to about 80 mg/kg, more preferably 1 mg/kg to about 50 mg/kg. In yet another embodiment, the dose ranges from about 1 mg to about 30 mg. Preferably, the effective dose is titrated under the supervision of a physician or medical care provider so that the optimum dose for the particular application is accurately determined. Thus, the present invention provides a dose suited to each individual patient.

Once the dosage range is established, a further advantage of the invention is that the patient can administer the norketamine with narcotic on an as-needed, dose-to-effect basis. Thus, the frequency of administration is under control of the patient. However, the relatively low dose with each administration will reduce the possibilities for abuse that arise under patient self-administration.

Yet another particular advantage of the present invention is that transmucosal or pulmonary administration of the norketamine with narcotic is non-invasive, and provides for introduction into the bloodstream almost as fast as i.v. administration, and much faster than perioral administration.

More importantly, a patient can control administration of the pain medication, because transmucosal or pulmonary administration provides for precise control over the dosage and effect of the drug used to offset changes in activity and pain levels throughout a day. Transmucosal or pulmonary administration of the norketamine/opioid compositions optimally provides for dose-to-effect administration of the drug. Transdermal administration, though not as fast acting, similarly allows for precise control of the dosage and also provides for excellent dose-to-effect administration of the drug.

Thus, according to the invention, the patient can safely administer an amount of drug effective to alleviate pain by controlling the amount and frequency of administration of a formulation according to the invention. Safe patient regulated control of pain medication is an important advantage because pain is such a subjective condition. The advantage is two-fold here, as the patient can effectively alleviate pain, and the power to alleviate the pain will have significant psychological benefits. A positive psychological attitude can significantly improve the course and outcome of a treatment regimen, as well as making the entire process more bearable to the patient.

The term “breakthrough pain” is used herein in accordance with its usual meaning in pain treatment. For example, breakthrough pain can refer to pain experienced by a subject receiving treatment for pain, but who experiences a level of pain that is not treatable by the current treatment regimen. “Spike pain” is an acute form of breakthrough pain: Usually medications or therapies for chronic pain do not provide adequate relief for breakthrough pain, either because the maximum pain relief effects of these regimens have been achieved, because of tolerance to medications that has developed, or because the treatment is not fast enough. Pain related to “wind up” is that pain arising from repeated stimuli which causes a temporal summation of C-fiber-mediated responses of dorsal horn nociceptive neurons and that may be expressed physically as hyperalgesia (increased pain sensation) and allodynia (pain arising from a stimulus that is not normally painful).

A subject in whom administration of norketamine/opioid compositions is an effective therapeutic regimen for management of pain, or for synergism with alternative pain therapy is preferably a human, but can be any animal. Thus, as can be readily appreciated by one of ordinary skill in the art, the methods and devices of the present invention are particularly suited to administration of norketamine/opioid compositions to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., i.e., for veterinary medical use. For veterinary use, rectal administration or transdermal administration are convenient and allow for minimal aggravation or irritation of the animal.

The term “mucosal” refers to a tissue comprising a mucous membranes, such as the oral, buccal, rectal, or vaginal mucosa and the pulmonary mucosa. “Transmucosal” refers to administration of a drug through the mucosa to the bloodstream for systemic delivery of the drug. One distinct advantage of transmucosal delivery is that it provides delivery of drug into the bloodstream almost as fast as parenteral delivery, but without the unpleasant necessity of injection.

The term “transdermal administration” in all its grammatical forms refers to administration of a drug through the dermis to the bloodstream for systemic delivery of the drug. The advantages of transdermal administration for drug delivery are that it does not require injection using a syringe and needle, it avoids necrosis that can accompany i.m. administration of drugs, it avoids the need to constantly suck on a lollipop, and transdermal administration of a drug is highly amenable to self administration.

“Pulmonary administration” refers to administration of a drug through the pulmonary tract (i.e., inhaled into the lungs) to the bloodstream for systemic delivery of the drug. The present invention contemplates pulmonary administration through an inhaler in a particular aspect.

The term “mucosal penetration enhancer” refers to a reagent that increases the rate or facility of transmucosal penetration of norketamine or a ketamine/norketamine prodrug, such as but not limited to, a bile salt, fatty acid, surfactant or alcohol. In specific embodiments, the permeation enhancer can be sodium cholate, sodium dodecyl sulphate, sodium deoxycholate, taurodeoxycholate, sodium glycocholate, dimethylsulfoxide or ethanol.

A “therapeutically effective amount” of a drug is an amount effective to demonstrate a desired activity of the drug. According to the instant invention, a therapeutically effective amount of a norketamine with narcotic is an amount effective to alleviate, i.e., noticeably reduce, pain in a patient.

The invention will now be described in greater detail, with particular reference to transdermal, transmucosal, and pulmonary administration of the norketamine/opioid compositions and additional therapeutically active drugs or agents with which the norketamine/opioid compositions can be administered.

### Pulmonary and Nasal Transmucosal Administration of Norketamine and Narcotic

The present invention contemplates formulations comprising norketamine/opioid compositions for use in a wide variety of devices that are designed for the delivery of pharmaceutical compositions and therapeutic formulations to the respiratory tract, preferably the pulmonary and bronchial passages. A preferred route of administration of the present invention is in an aerosol spray for pulmonary inhalation. Norketamine/opioid compositions, optionally combined with a dispersing agent, or dispersant, can be administered in an pulmonary formulation as a dry powder or in a solution or suspension, optionally with a diluent.

As used herein, the term “aerosol” refers to suspension in the air. In particular, aerosol refers to the particalization or atomization of a formulation of the invention and its suspension in the air. According to the present invention, a pulmonary formulation is a formulation comprising a norketamine/opioid compositions for inhalation or pulmonary administration.

As used herein, the term “inhaler” refers both to devices for nasal-transmucosal and pulmonary administration of a drug, e.g., in solution, powder and the like. For example, the term “inhaler” is intended to encompass a propellant driven inhaler or a dry powder inhaler, such as is used for to administer antihistamine for acute asthma attacks, and plastic spray bottles, such as are used to administer decongestants. As used herein, “inhaler” will also encompass the term “nebulizer” as it is well known in the art.

As used herein, the term “dispersant” refers to an agent that assists aerosolization or absorption of the norketamine/opioid compositions in mucosal tissue, or both. In a specific aspect, the dispersant can be a mucosal penetration enhancer. Preferably, the dispersant is pharmaceutically acceptable. As used herein, the term “pharmaceutically acceptable” means approved by a regulatory agency of the Federal or a state government or listed in the U.S. Pharmacopeia or other generally recognized pharmacopeia for use in animals, and more particularly in humans.

Suitable dispersing agents are well known in the art, and include but are not limited to surfactants and the like. For example, surfactants that are generally used in the art to reduce surface induced aggregation of norketamine or a ketamine/norketamine prodrug caused by atomization of the solution forming the liquid aerosol may be used. Nonlimiting examples of such surfactants are surfactants such as polyoxyethylene fatty acid esters and alcohols, and polyoxyethylene sorbitan fatty acid esters. Amounts of surfactants used will vary, being generally within the range or 0.001 and 4% by weight of the formulation. Suitable surfactants are well known in the art, and can be selected on the basis of desired properties, depending on the specific formulation, concentration of norketamine and narcotic, diluent (in a liquid formulation) or form of powder (in a dry powder formulation), etc.

The liquid formulations contain norketamine/opioid compositions, optionally with a dispersing agent, in a physiologically acceptable diluent. The dry powder formulations of the present invention consist of a finely divided solid form of norketamine/opioid compositions, optionally with a dispersing agent. With either the liquid or dry powder formulation, the formulation must be aerosolized. That is, it must be broken down into liquid or solid particles in order to ensure that the aerosolized dose actually reaches the mucous membranes of the bronchial passages or the lungs. The term “aerosol particle” is used herein to describe the liquid or solid particle suitable for transmucosal or pulmonary administration, i.e., that will reach the mucous, membranes or lungs. Other considerations, such as construction of the delivery device, additional components in the formulation, and particle composition and characteristics are important. These aspects of transmucosal or pulmonary administration of a drug are well known in the art, and manipulation of formulations, aerosolization means, and construction of a delivery device require, at most, routine experimentation by one of ordinary skill in the art.

For nasal or pulmonary administration, a useful device is a small, hard bottle to which a metered dose sprayer is attached. In one embodiment, the metered dose is delivered by drawing the norketamine and/or ketamine/norketamine prodrug solution into a chamber of defined volume, which chamber has an aperture dimensioned to aerosolize the formulation by forming a spray when a liquid in the chamber is compressed. The chamber is compressed to administer the norketamine and narcotic. In a specific embodiment, the chamber is a piston arrangement. Such devices are commercially available.

Alternatively, a plastic squeeze bottle with an aperture or opening dimensioned to aerosolize an pulmonary formulation by forming a spray when squeezed. The opening is usually found in the top of the bottle, and the top is generally tapered to partially fit in the nasal passages for efficient administration of the aerosol formulation. Preferably, the nasal or pulmonary inhaler will provide a metered amount of the formulation, for administration of a measured dose of the drug.

Often, the aerosolization of a liquid or a dry powder formulation for inhalation into the lung will require a propellent. The propellent may be any propellant generally used in the art. Specific nonlimiting examples of such useful propellants are a chlorofluorocarbon, a hydrofluorocarbon, a hydrochlorofluorocarbon, or a hydrocarbon, including trifluoromethane, dichlorodifluoromethane, dichlorotetrafluoroethanol, and 1,1,1,2-tetrafluoroethane, or combinations thereof.

Systems of aerosol delivery, such as the pressurized metered dose inhaler and the dry powder inhaler are disclosed in Newman, S. P., Aerosols and the Lung, Clarke, S. W. and Davia, D. editors, pp. 197-222, and in U.S. Pat. Nos. 6,358,530, 6,360,743, 6,406,745, 6,423,683, 6,565,888, and 6,630,169, the disclosures of which are incorporated herein in their entireties, and can be used in connection with the present invention.

In a further embodiment, as discussed in detail infra, a nasal transmucosal or pulmonary formulation of the present invention can include other therapeutically or pharmacologically active ingredients in addition to norketamine/opioid compositions, such as but not limited to a benzodiazepine or a narcotic analgesic.

With regard to construction of the delivery device, any form of aerosolization known in the art, including but not limited to spray bottles, nebulization, atomization or pump aerosolization of a liquid formulation, and aerosolization of a dry powder formulation, can be used in the practice of the invention.

As noted above, in a preferred aspect of the invention, the device for aerosolization is a metered dose inhaler. A metered dose inhaler provides a specific dosage when administered, rather than a variable dose depending on administration. Such a metered dose inhaler can be used with either a liquid or a dry powder formulation. Metered dose inhalers are well known in the art.

### Transmucosal Administration

As noted above, the present invention is directed inter alia to transmucosal administration of norketamine with an opioid. Initial studies demonstrate that nasal administration of the drugs, either via the nasal mucosa or pulmonary inhalation and absorption via pulmonary mucosa, is highly effective for the treatment of pain. Subsequently, it has been discovered that other routes of transmucosal administration of the drug combinations are also effective for treatment of pain, as set forth above. In particular, it has surprisingly been discovered that transmucosal administration of the drugs allows for effective pharmacokinetics with low doses of the drug, thus avoiding dysphoria or other side effects associated with bolus i.v. or i.m. dosing. Transmucosal norketamine with narcotic is particularly indicated for breakthrough and spike pain, e.g., as described in greater detail above.

According to the invention, any transmucosal route of administration, including but not limited to rectal, oral, vaginal, buccal, etc. can be employed. In particular, the present invention is directed to the following transmucosal routes of administration. It can be readily appreciated that any of the transmucosal routes of administration may be enhanced by use of a mucosal penetration enhancer, e.g., as described supra. The selection of a particular mucosal penetration enhancer may depend on the characteristics of the specific mucosa. These factors are addressed in greater detail below.

### Administration Via Suppositories

In another aspect, norketamine and narcotic are formulated in a matrix suitable for rectal (or vaginal) insertion, i.e., in a suppository. The invention is not limited to any particular suppository formulation. Indeed, many suppository formulations are known in the art, e.g., as described in Remington's Pharmaceutical Sciences, Physician's Desk Reference, and U.S. Pharmacopeia. Administration via suppositories may be preferred in certain situations, e.g., because convention and custom prefers it, or where nasal administration is deemed unacceptable.

### Administration Via Buccal Patch

According to the invention, norketamine and an opioid can be formulated in a buccal patch for administration via the interior of the cheek. It may be appreciated that a buccal patch constitutes another form of transmucosal administration. The technology for preparing buccal patch formulations is known in the art, e.g., Remington's Pharmaceutical Sciences, supra.

### Oral-Pharyngeal Administration

In yet another embodiment, the norketamine and opioid can be formulated for oral-pharyngeal, including sublingual and transbuccal, administration. For example, norketamine/opioid compositions can be incorporated in a “candy” matrix, such as that described in U.S. Pat. No. 4,671,953, in a gum base, or a lozenge. In another embodiment, the norketamine/opioid compositions can be formulated in a capsule or pill form for sublingual placement.

It is particularly contemplated that norketamine/opioid compositions for oral-pharyngeal administration may be formulated with a flavor masking agent or coating. Many flavor masking agents for use with oral pharmaceuticals are known in the art and can be selected for use with the present invention.

### Oral Administration

In still a further embodiment, the norketamine and opioid be formulated for oral administration via the stomach and intestinal mucosa. For oral administration, the drug can be administered in a carrier designed for drug release in either the stomach (an 43 acidic environment), or the intestines, or both. Many capsules, pills, and matrices for oral administration of a drug are known in the art, and can be selected on the basis of compatibility with norketamine and narcotic and the desired point and rate of drug release by the ordinary skilled physician. Sustained release formulations are preferred. One of skill in the art will appreciate that dosages for oral administration are generally higher than dosages administered by a parenteral route.

### Transdermal Administration

In a further embodiment, as noted above, the present invention is directed to transdermal administration of norketamine with a narcotic. It has been discovered that transdermal administration is also effective for treatment of pain, as set forth above, for many of the same reasons transmucosal administration is effective. In particular, it has surprisingly been discovered that transdermal administration of norketamine and opioid compositions allows for effective pharmacokinetics with low doses of the drug, thus avoiding dysphoria or other side effects associated with bolus i.v. or i.m. dosing. Transdermal administration is particularly indicated for breakthrough and spike pain, e.g., as described in greater detail above.

Various and numerous methods are known in the art for transdermal administration of a drug, e.g., via a transdermal patch. These methods and associated devices provide for control of the rate and quantity of administration of a drug, and some allow for continuous modulation of drug delivery. Transdermal patches are described in, for example, U.S. Pat. No. 5,407,713, issued Apr. 18, 1995 to Rolando et al.; U.S. Pat. No. 5,352,456, issued Oct. 4, 1004 to Fallon et al; U.S. Pat. No. 5,332,213 issued Aug. 9, 1994 to D'Angelo et al; U.S. Pat. No. 5,336,168, issued Aug. 9, 1994 to Sibalis; U.S. Pat. No. 5,290,561, issued Mar. 1, 1994 to Farhadieh et al.; U.S. Pat. No. 5,254,346, issued Oct. 19, 1993 to Tucker et al.; U.S. Pat. No. 5,164,189, issued Nov. 17, 1992 to Berger et al; U.S. Pat. No. 5,163,899, issued Nov. 17, 1992 to Sibalis; U.S. Pat. Nos. 5,088,977 and 5,087,240, both issued Feb. 18, 1992 to Sibalis; U.S. Pat. No. 5,008,110, issued Apr. 16, 1991 to Benecke et al; and U.S. Pat. No. 4,921,475, issued May 1, 1990 to Sibalis, the disclosure of each of which is incorporated herein by reference in its entirety.

It can be readily appreciated that a transdermal route of administration may be enhanced by use of a dermal penetration enhancer, e.g., such as enhancers described in U.S. Pat. No. 5,164,189 (supra), U.S. Pat. No. 5,008,110 (supra), and U.S. Pat. No. 4,879,119, issued Nov. 7, 1989 to Aruga et al., the disclosure of each of which is incorporated herein by reference in its entirety.

In another embodiment, the norketamine/opioid compositions can be delivered in a vesicle, in particular a liposome (see Langer, 1990, Science 249:1527-1533; Treat et al, 1989, in Liposomes in the Therapy of Infectious Disease and Cancer, Lopez-Berestein and Fidler (eds.), Liss: New York, pp. 353-365; Lopez-Berestein, ibid, pp. 317-327; see generally ibid). To reduce its systemic side effects, this may be a preferred method for introducing norketamine/opioid compositions.

In yet another embodiment, norketamine and opioid may be delivered in a controlled release system. For example, the drugs may be administered using intravenous infusion, an implantable osmotic pump, a transdermal patch, liposomes, or other modes of sustained release administration. In one embodiment, a pump may be used (see Langer, supra; Sefton, 1987, CRC Crit. Ref. Biomed. Eng. 14:201; Buchwald et al, 1980, Surgery 88:507; Saudek et al, 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). Other controlled release systems are discussed in the review by Langer (1990, Science 249:1527-1533).

### Additional Therapeutically Active Drugs or Agents

As note above, the invention contemplates coordinate administration of norketamine with an opioid, preferably, morphine. The invention provides a method of alleviating pain by the administration of both norketamine with an opioid where the dose of the norketamine, alone, would have been sub-optimal for pain treatment. As well, in a more preferred embodiment, invention provides a method of alleviating pain by the administration of both norketamine with an opioid where the dose of the opioid, alone, would have been sub-optimal for pain treatment. The invention takes advantage of the discovery that use of otherwise sub-optimal doses of norketamine works synergistically with a narcotic, in combination, to boost the analgesic effect of the combined therapy. However, other drug(s) may be used in addition to the described compositions.

For example, co-administration of the norketamine/opioid compositions with a benzodiazepine is indicated to counteract the potential dysphoric or hallucinogenic effects of high dose administration of norketamine/opioid compositions. Thus, a therapeutically effective amount of a benzodiazepine is an amount effective to inhibit dysphoria. In a further embodiment, an amount of a benzodiazepine also effective to sedate the patient may be administered.

The mild adverse effects of ketamine, e.g., dysphoria and/or hallucinations, sometimes called “ketamine dreams,” can occur upon administration of a dose of greater than 50 mg of ketamine, but usually require doses greater than 100 mg per kg of ketamine. One advantage of the present invention is that delivery of norketamine/opioid compositions allows for control of the dose to a level effective for analgesia, but below the level that results in dysphoria. Another is that norketamine/opioid compositions are less prone to adverse psychological effects than ketamine alone. However, it is possible that an individual may overdose, particularly in response to an acute episode of pain. Thus, co-administration of a benzodiazepine may be indicated in certain circumstances.

Benzodiazepines that may be administered according to the present invention include, but are not limited to, flurazepam (Dalmane), diazepam (Valium), and, preferably, Versed. In a preferred aspect, the transmucosal formulation of the invention comprises ketamine and a benzodiazepine, each present in a therapeutically effective amount.

Where medical necessity or preference dictates, parenteral administration of norketamine/opioid compositions can be effected to synergistically treat pain with other pain therapies. Alternate pain therapies include non-pharmaceutical treatments, such as but not limited to, chiropractic medicine, acupuncture, biofeedback, and other alternative therapies.

Preferably, the synergistic effects of norketamine and narcotic administration are reflected by reduced dependency on other pain therapies, or by an reduction in the level of pain experienced, or both. This aspect of the invention is based on the surprising discovery administration of norketamine/opioid compositions allow for a reduction over time of narcotic analgesics. Such a reduction over time runs counter to the normal course of pain treatment, where progressively larger doses of analgesics, particularly narcotic analgesics, are required to overcome tolerance.

Usually, combinations of pain medications yield at best additive or supplemental results. Thus, it is a significant advantage of the present invention that it allows for a reduction in the level of a pain medication, without compromising the level of pain relief.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

### EXAMPLES

### Example 1

Table of Some Embodiments of Norketamine and Opioid Compositions

| Norketamine | Opioid |
|---|---|
| R,S-Norketamine | Morphine |
| R-Norketamine | Morphine |
| S-Norketamine | Morphine |
| R,S-Norketamine | Codeine |
| R-Norketamine | Codeine |
| S-Norketamine | Codeine |
| R,S-Norketamine | Fentanyl |
| R-Norketamine | Fentanyl |
| S-Norketamine | Fentanyl |
| R,S-Norketamine | Methadone |
| R-Norketamine | Methadone |
| S-Norketamine | Methadone |
| R,S-Norketamine | Buprenophene |
| R-Norketamine | Buprenophene |
| S-Norketamine | Buprenophene |

### Example 2

Sprague Dawley (about 90 days old; 350 g) male rats were used (8 rats/drug/experimental group). R,S-norketamine, S-norketamine, R-norketamine (Yaupon Therapeutics Inc.) and R,S-ketamine (Sigma) were dissolved in saline and injected intraperitoneally (IP, 1 ml/kg). Each rat received four doses of a drug (1, 2, 4, 8 mg/kg; repeated block Latin square design; 48 h intervals). Saline served as control.

A sciatic nerve constriction model of peripheral neuropathy previously employed was used [Benett and Xie, 1988]. Briefly, under pentobarbital anesthesia (40 mg/kg, IP) the ligation of sciatic nerve and sham surgery were performed on the left and right hind paws, respectively. Proximal to the sciatic trifuracation, nerve (7 mm) was freed from adhering tissue and four loose ligatures were tied around nerve (1 mm apart) with 4.0 chromic gut, barely constricting the diameter of the nerve. The incision was closed in layers. Rats showed a mild aversion of the affected paw and a mild degree of foot drop. No severe motor impairment was observed.

The analgesic properties of drugs were determined in neuropathic rats. Responsiveness to both mechanical and thermal noxious stimulations was determined in separate groups of rats. Rats were trained on three occasions before initiation of the study. Experiments were performed on days 7, 9, 11 and 14 of recovery from surgery (as this is the time of maximal hyperalgesia [Holtman et al., 2003]). Responses were assessed prior to (baseline, taken twice) and at 15-120 min after injection. The left and right paws were tested alternatively in each rat.

Mechanical hyperalgesia was measured using an increasing amount of weight to the paw [Randall and Selitto, 1957]. The hind paw was placed between a flat surface and a blunt pointer in the Basile Analgesimeter (UGO Basile) and increasing pressure (32 g/s) was applied to the dorsal side of the paw. Vocalization was used as end-point [vocalization threshold, VT (g)]. Cut-off at 300 g prevented tissue damage.

The thermal hyperalgesia was measured by plantar test which used a ramp heat stimulus [Hargreaves et al., 1988]. The radiant heat (60% intensity) was positioned under the glass floor directly beneath the plantar hind paw in Plantar Stimulator Analgesia Meter (IITC, Life Science). Latency of paw withdrawal from the heat source was measured [paw withdrawal threshold, PWT (s)]. A cut-off at 20 s prevented tissue damage.

The behavioral effects of drugs were determined in intact (unoperated) rats. Locomotor activity was determined using the Opto-Varimex infrared photocell-based activity monitor (Columbus Instrument). All activities were scored during 5 min sessions, prior to and 15, 60 and 120 min after injection. All testing were conducted between 10:00-13:00. Assessments were performed in 48 h intervals. Ataxia was determined at 0, 15, 10 and 15 min after injection. The modified behavioral scale [Sturgon et al., 1979] was used for quantification (Table 1).

TABLE 1
Behavioral Rating Scale

| Rating | Ataxia |
|---|---|
| 0 | Coordinated movement |
| 1 | Loss of balance when rearing, jerky movement |
| 2 | Frequent falling to side with attempted walking |
| 3 | Unable to walk |

All data were normalized for preinjection baseline values. Areas under the curves (AUC) were calculated for normalized data. Maximum possible effect was calculated as % MPE=(post drug response−baseline/cut off−baseline)×100. ED₅₀ was calculated from % MPE vs. log dose curves. All data were presented as mean ±SEM of 8 rats. The statistical analysis was performed with use of one and two-way repeated measures analysis of variance (ANOVA), post hoc Student Newman Keulus (SNK), Dunnan and t tests.

### R,S-Norketamine Produces Dose-Related Antinociception in Rodent Model Of Neuropathy (Mechanical and Thermal Tests).

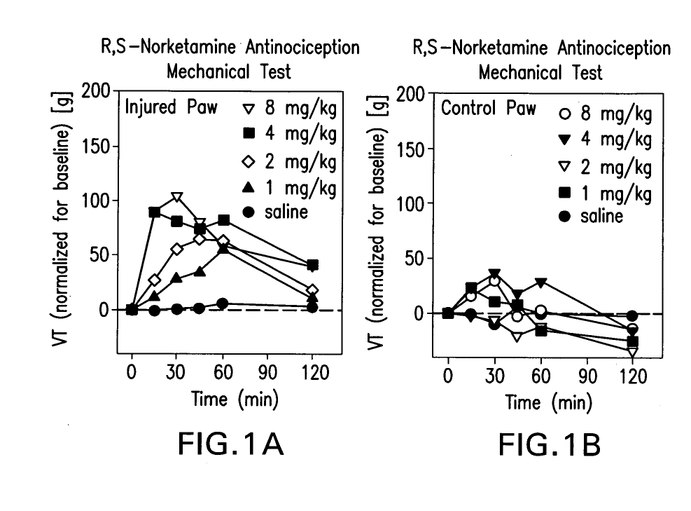

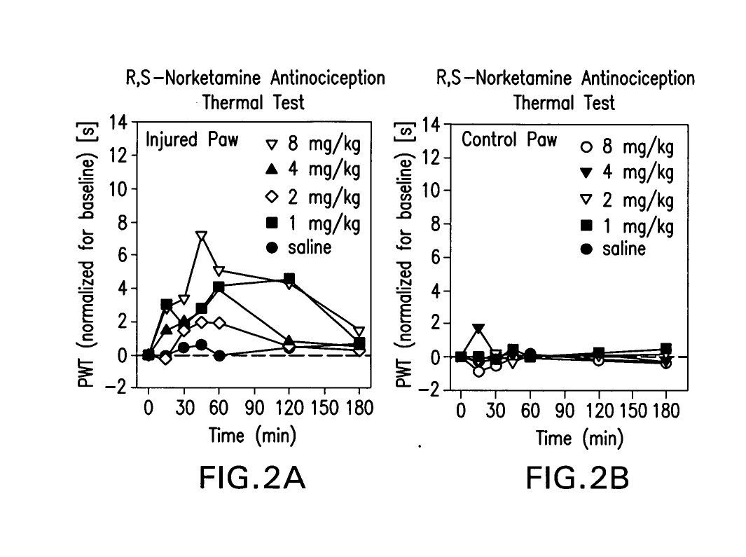

The racemic mixture of norketamine produced dose-related antinociception in response to both mechanical and thermal noxious stimuli on the nerve-injured paw. The effect was of rapid onset and moderate duration (>2 h). No antinociception was observed on the control paw (sham-operated) [FIG. 1A,B and FIG. 2A,B]. Saline had no effect on both paws. These data demonstrate that the major metabolite of ketamine, norketamine, attenuated in a dose-related fashion the enhanced sensitivity to mechanical and thermal noxious stimuli (mechanical and thermal hyperalgesia) in neuropathic rats. This suggests that norketamine blocks NMDA receptor-mediated sensitization following peripheral nerve injury.

### R,S-Norketamine has a Similar Antinociceptive Efficacy as R,S-Ketamine in Rodent Model of Neuropathy.

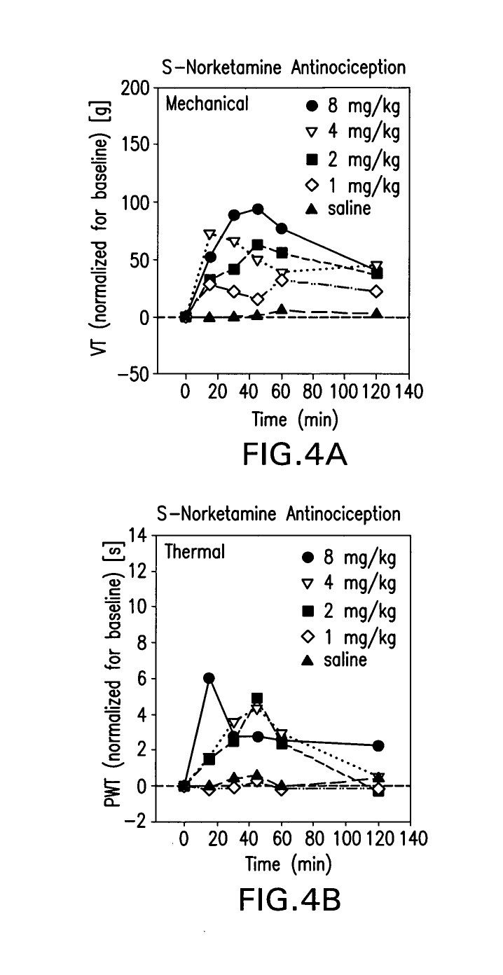

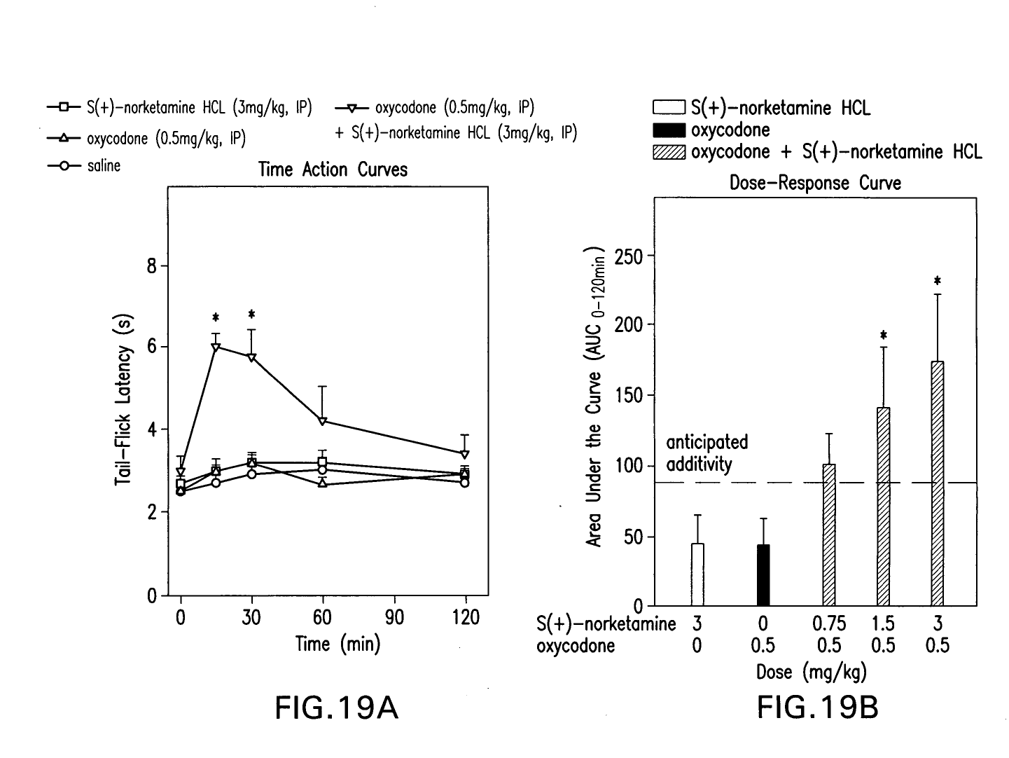

The antinociceptive potencies of R,S-norketamine and R,S-ketamine were similar on nerve injured paw (ED₅₀=11.3±0.23 and 15.8±0.38 mg/kg, respectively) [FIGS. 4, 19]. This suggests that a major metabolite, norketamine, contributes significantly to the antinociceptive effect of a parent drug, ketamine.

### S-Norketamine Produced the Greater Antinociceptive Effect than R-Norketamine in Rodent Model of Neuropathy.

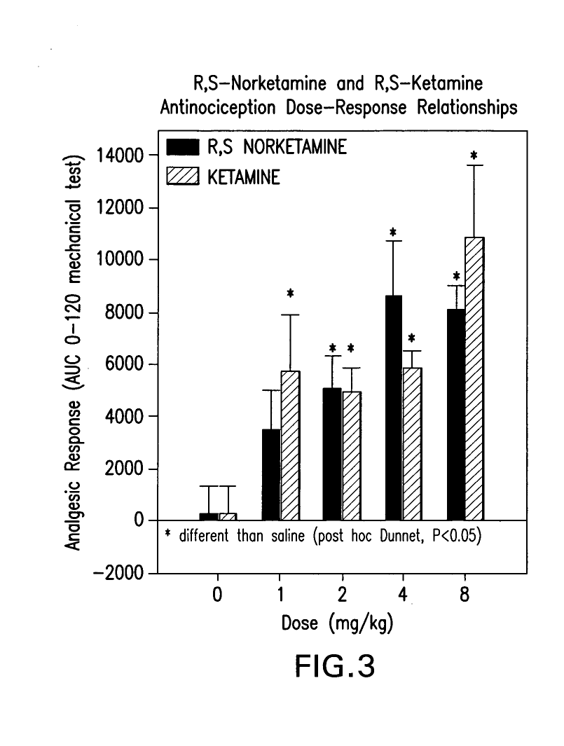

The S and R enantiomers of norketamine attenuated, in dose-related fashion, the mechanical and thermal hyperalgesia on the nerve-injured paw [FIG. 3A,B and FIG. 4A,B]. Neither drug had effect on sham-operated paw (data not shown). The antinociceptive efficacy was markedly greater for S-norketamine compared to R-norketamine (ED₅₀=7.3±0.18 and 51.1±0.54 mg/kg, respectively).

### There is a Good Correlation Between the Time Courses of Antinociception and Plasma Levels of S-Norketamine.

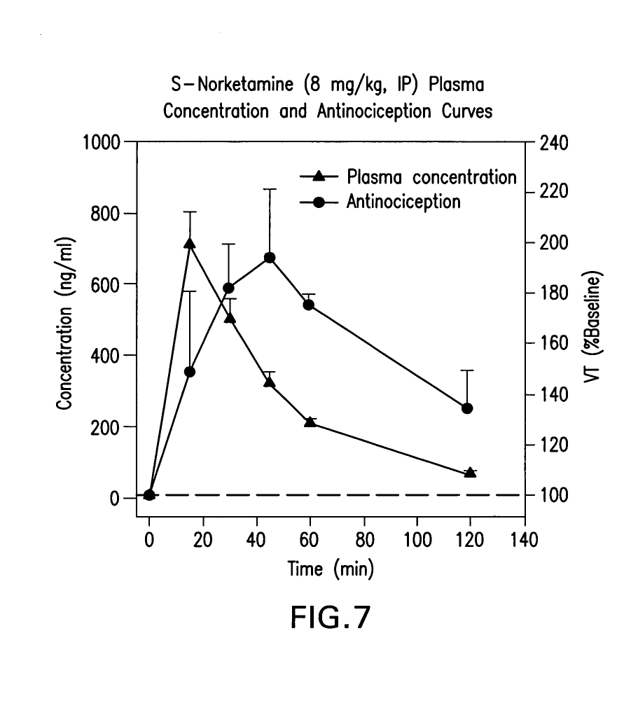

A pilot studies demonstrated that the time curse of plasma levels of S-norketamine paralleled the time action curve for antinociception [FIG. 7]. Plasma concentration of S-norketamine 200-700 ng/ml was associated with the significant analgesic effect after IP administration in rat.

Unoperated (intact) rats were used to determine whether excitatory, depressive or no motor effects are observed at doses that showed the antinociceptive effect in neuropathic rats.

### Norketamine has Less Effect on the Activity Level than Ketamine.

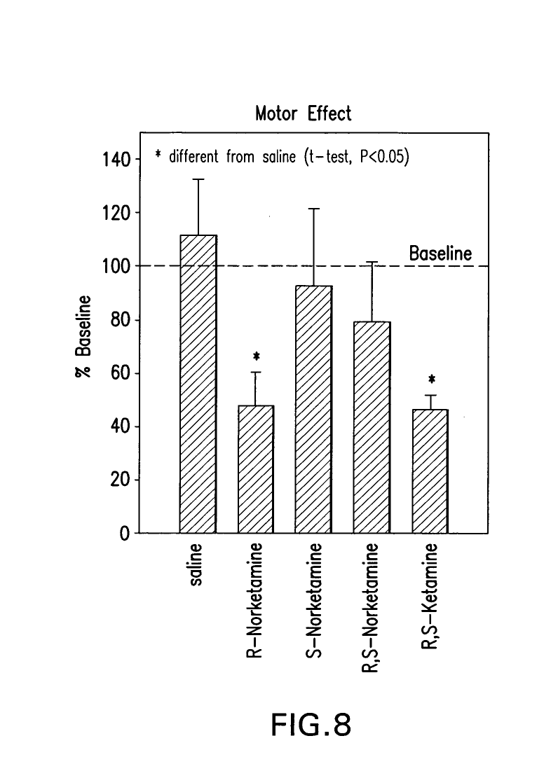

The effects of norketamine and ketamine on activity level were dose-related (data not shown). As can be seen, at the highest dose (8 mg/kg), the motor effect (depressive) was less pronounced for R,S-norketamine compared to R,S-ketamine. Further, the locomotor effect was less for S than R isomer of norketamine [FIG. 8].

### Norketamine does not Induce Ataxia in Rats.

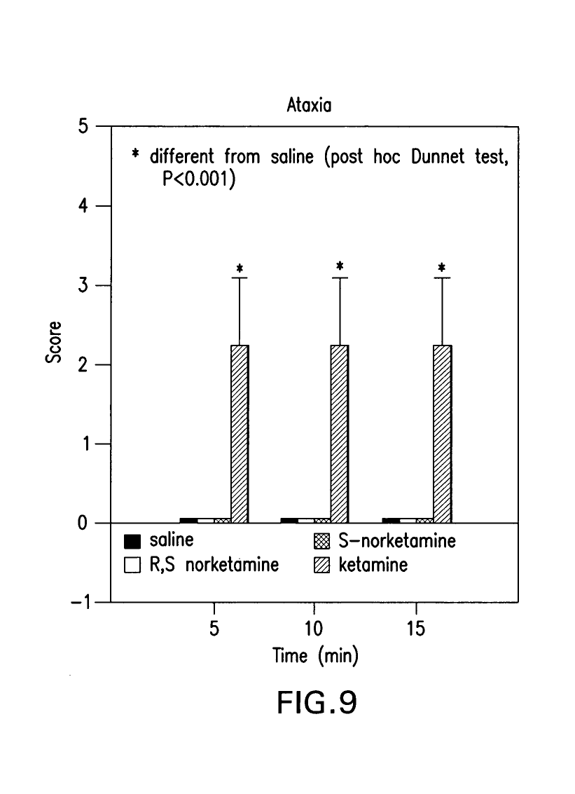

The pilot study demonstrated no ataxia after administration of R,S- or S-norketamine in rats. This was in contrast to marked ataxia produced by R,S-ketamine [FIG. 9]. These data suggest that ketamine-induced ataxia is not due to its metabolite, norketamine.

These studies demonstrated that: 1) R,S-Norketamine and R,S-ketamine have similar same-dose effects in a rodent model of peripheral neuropathy (mechanical and thermal hyperalgesia). The analgesic properties of R,S-norketamine are mostly residing in the S isomer. The R isomer appears to be a less potent analgesic drug. 2) The effect on motor performance and sedation is less pronounced for R,S-norketamine compared to R,S-ketamine. The locomotor effect of norketamine seems to be due to R enantiomer. Taken together, S-norketamine appears to have an equal antinociceptive efficacy but better side effects profile than clinically used ketamine. This initial feasibility study provided a basis for phase II preclinical and clinical studies to further characterize norketamine enantiomers.

### Example 3

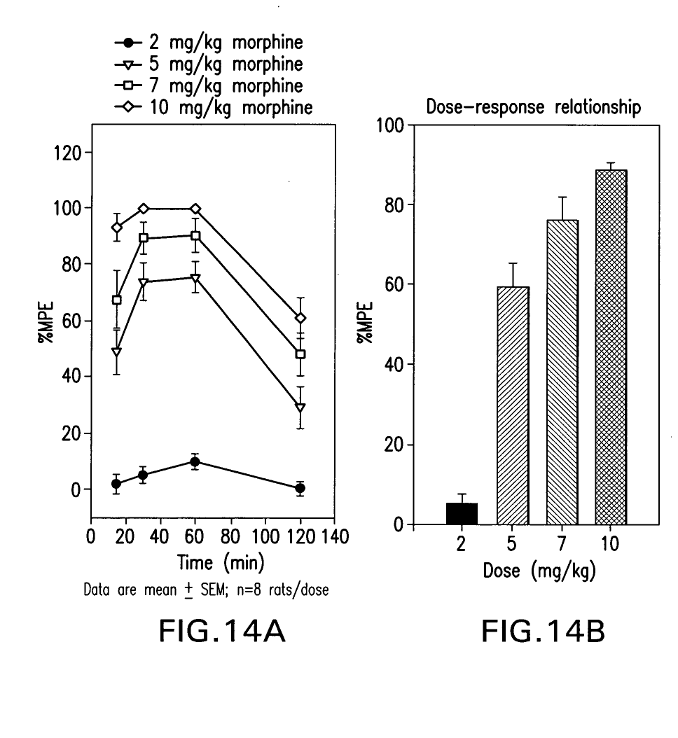

A study was undertaken to determine whether S-norketamine (“norKET”) enhances the analgesic effect of morphine (“MOR”). (The side effect profile was determined to be better for the S than the R enantiomer.) Both drugs were given alone and in combination by intraperitoneal [IP; S-norketamine=0.75, 1.5, 3 mg/kg and MOR=3 mg/kg] or intrathecal [IT; S-norKET=10, 50, 100 mcg and MOR=0.5 mcg)] routes in male Sprague-Dawley rats. Saline (vehicle) served as a control. Responsiveness to thermal noxious stimuli was determined using the tail-flick assay (baseline tail-flick latency (TFL) ˜2-3 s; cut off TFL=10 s). TFL was determined at 0, 15, 30, 60, 90, and 120 min. Data demonstrated that S-norKET, in doses that do not produce an antinociceptive effect alone, dose-dependently potentiated the antinociceptive effect of MOR in rats. Significant analgesic interaction was observed after co-administration of MOR and S-norKET either IP or IT (FIGS. 14-18).

Male Sprague-Dawley rats, approximately 90 days old, weighting about 300 g were used. Intrathecal catheter: Chronic catheterization of the spinal subarachnoid space was performed according to Yaksh and Rudy (1976). Drugs: Morphine sulfate (Mallinckrodt) and S-norketamine hydrochloride (Yaupon Therapeutics, Inc.) were dissolved in saline. Saline served as control. Doses refer to salt forms.

Graded doses of morphine and S-norketamine alone as well as a fixed dose of morphine combined with various doses of S-norketamine were administered intrathecally (IP) or intrathecally (IT) in volumes equal to 1 ml/kg and 10 μl, respectively. Doses were balanced by Latin square design: 2× (4×4). Injections were made at weekly intervals.

Doses of morphine and S-norketamine administered alone and in combination. Drugs were administered by intraperitoneal (IP) or intrathecal (IT) routes in rats. Saline (dose 0) served as control. See Table 2, below.

| | IP (mg/kg) | | | | IT (μg) | | | |
|---|---|---|---|---|---|---|---|---|
| Morphine | 2 | 5 | 7 | 10 | 0 | 3 | 10 | 30 |
| Norketamine | 0 | .75 | 1.5 | 3 | 0 | 10 | 50 | 100 |
| Morphine + Norketamine | 0 | .75 | 1.5 | 3 | 0 | 10 | 50 | 100 |

Tail flick latencies (TFL) were measured using a standard tail-flick apparatus (LifeScience). Preinjection baseline and cut-off times were equal to 2-3 s and 10 s, respectively. TFL was assessed twice prior to (baseline) and at fixed time points after injection. All data were normalized for baseline. The areas under the time action curves (AUC0-120 min) were calculated for normalized data. The percent of maximum effect was calculated as % MPE=[(TFL-baseline)/(10-baseline)×100] at each time point. Data are presented as mean ±SEM of (n) rats. Data were analyzed by two-way ANOVA and post-hoc Student-Newman-Keuls (SNK) method. Level of significance was P≦0.5.

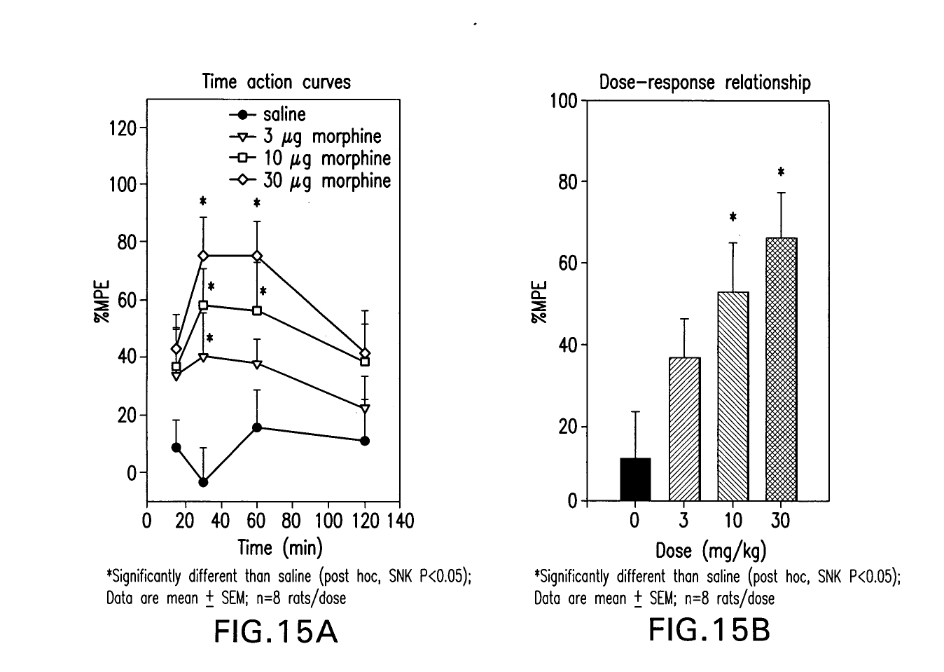

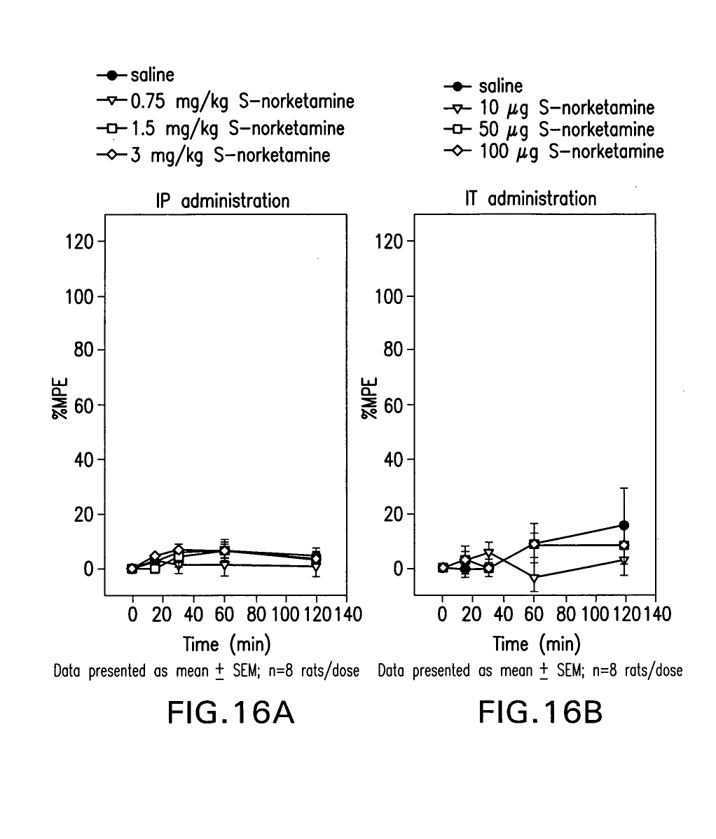

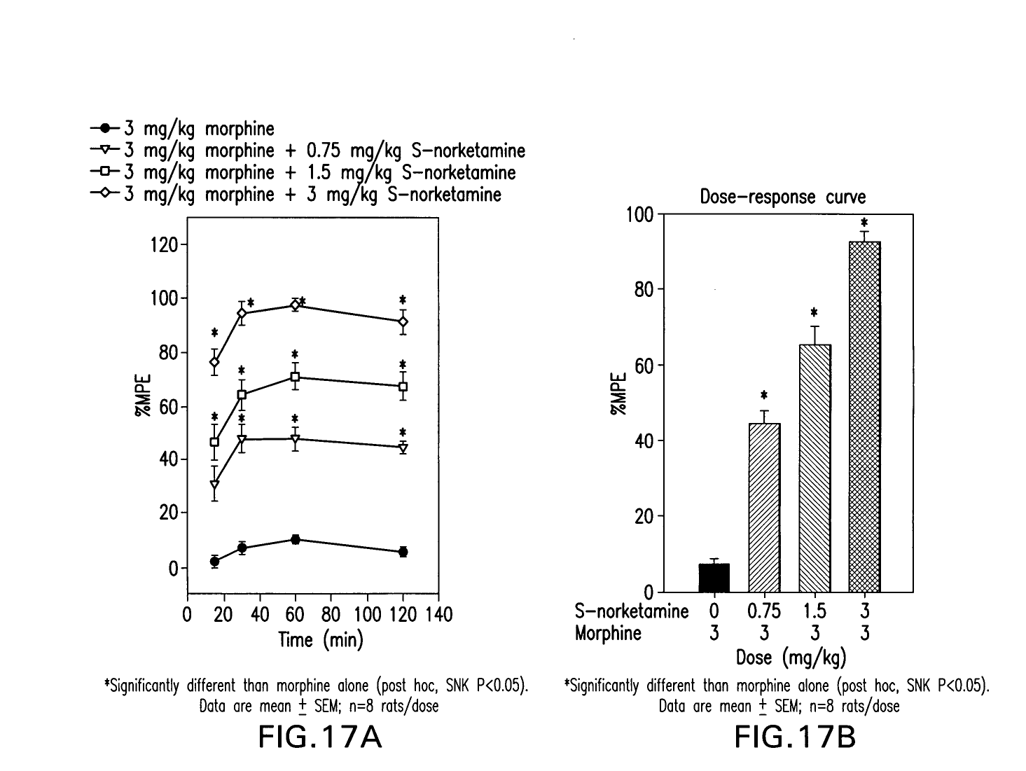

Morphine produced dose-related antinociception in response to radiant thermal stimulus (tail-flick test) both after IP (2-10 mg/kg) [FIG. 14] and IT (3-30 μg) administration in rats [FIG. 15]. S-Norketamine did not produce an antinociceptive effect after IP (0.75-3 mg/kg) [FIG. 16A] or IT (10-100 μg) [FIG. 16B] administration in rats (tail-flick test). S-Norketamine, in IP doses that do not produce an antinociceptive effect alone (0.75-3 mg/kg, IP), dose-dependently potentiated the antinociceptive effect of a low dose morphine (3 mg/kg; IP) [FIG. 17]. Morphine (3 mg/kg, IP) in combination with S-norketamine (3 mg/kg; IT) produced the maximum antinociceptive effect (% MPE=100%). The effect of this magnitude (% MPE=100%) was achieved after administration of approximately three-fold higher dose of morphine alone (10 mg/kg, IP) [FIG. 17B vs. FIG. 14B]. The time action curves appear to be longer after morphine plus S-norketamine than morphine alone (IP route) [FIG. 17A vs. FIG. 14A].

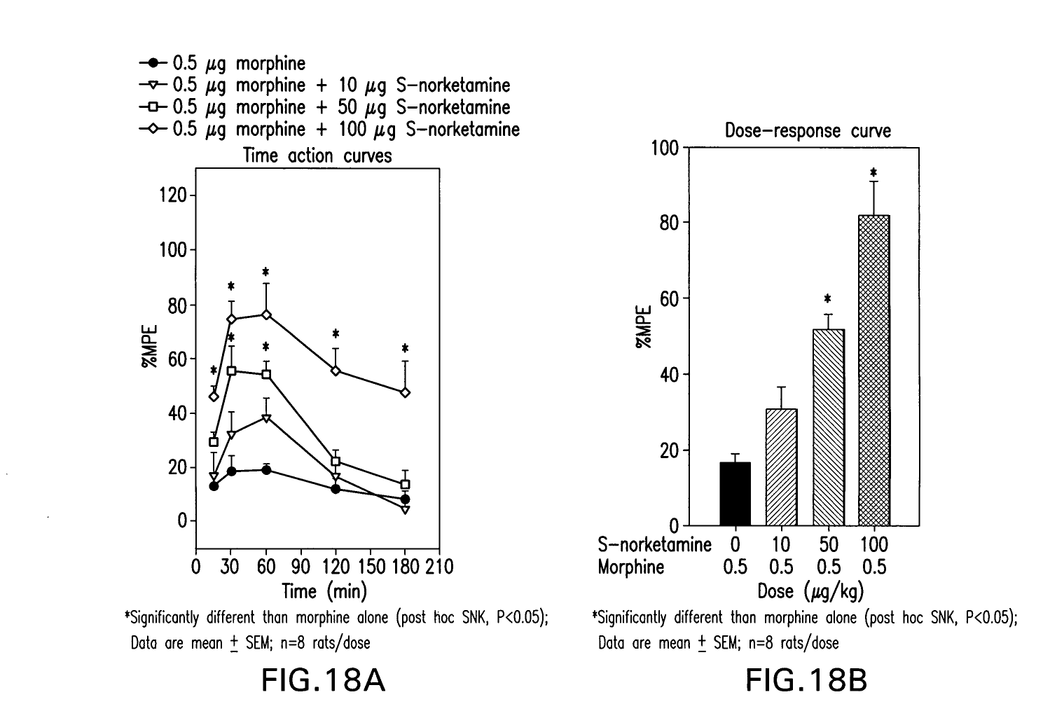

S-Norketamine, in IT doses that do not produce an antinociceptive effect alone (10-100 μg, IT), dose-dependently potentiated the antinociceptive effect of a low dose morphine (0.5 μg; IT) [FIG. 18]. Morphine (0.5 μg, IT) in combination with S-norketamine (100 μg, IT) produced greater antinociceptive effect (% MPE=80%) than this produced by the sixty-fold higher dose (30 μg, IT) of morphine alone (% MPE=60%) [FIG. 18B vs. FIG. 18B].

A likely synergistic antinociceptive interaction was observed after coadministration of morphine and S-norketamine either by peripheral (IP) or central (IT) routes in rats. The ability of S-norketamine to enhance morphine analgesia (IP) was greater than that previously demonstrated for ketamine-morphine (IP) interaction in rats (Holtman et al., 2003). These findings are of importance in the development of a novel NMDA receptor antagonist and opioid receptor agonist combination therapy for pain management, in particular neuropathic pain.

### Example 4

### Hydrolysis Study Protocol for Norketamine Prodrug

Stability studies were conducted both in Hanks' buffer of pH 7.4 and human plasma over a period of 48 hrs (n=3). From the stock solution of 1 mg/ml of norketamine esters and norketamine in acetonitrile, a series of standard solutions in the concentration range of 50-1000 ng/ml with acetonitrile were prepared. Hanks' buffer (300 μL) and plasma (200 μL) were spiked with 10 μL of different concentrations of both the drug solutions. The Hanks' buffer samples were vortexed for 30 sec and centrifuged (20 min at 12000 rpm) and the supernatant is transferred to HPLC vials.

In the case of the plasma samples, 750 μL of acetonitrile was added and vortexed for 30 sec and centrifuged (20 min at 12000 rpm) and the supernatant removed. The supernatant was evaporated at 37° C. under nitrogen and reconstituted with 400 μL acetonitrile and transferred to HPLC vials. The HPLC system consisted of a Perkin Elmer series 200 autosampler and pump and a 785A UV/VIS detector with Turbochrome 6.1 software. A reversed phase 220×4.6 mm Brownlee Spheri 5 VL C-18 5μ column and a guard column were used. The detector was set at a wavelength of 215 nm. The mobile phase consisted of 0.1% trifluoroacetic acid (adjusted to pH 3 with triethylamine+0.1% sodium heptane sulfonate and 5% acetonitrile) acetonitrile: (25:75) at a flow rate of 1.5 ml/min. Injection volume was 100 μL and run time was 10 minutes.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

### Example 5

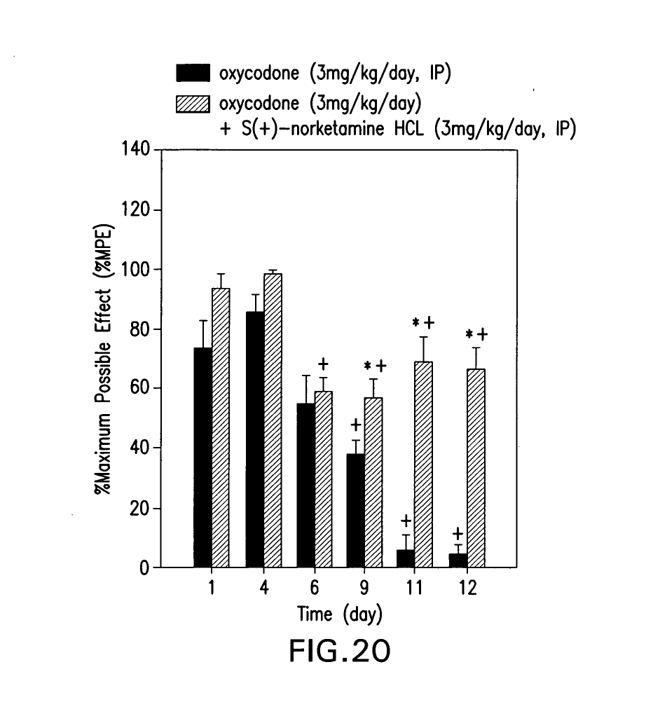

FIG. 19 demonstrates an analgesic response of S-norketamine HCl and oxycodone alone and in combination. A tail flick test as described above was administered intrapertoneally to eight Sprague-Dawley rats as described above. The * symbol denotes a statistically difference with oxycodone and norketamine combined versus the two drugs used alone. Data were analyzed with the SNK method, with P less than 0.05. FIG. 20 illustrates attenuation of oxycodone tolerance development by administration with S-norketamine HCl. A tail flick test as described above was administered intrapertoneally to eight Sprague-Dawley rats as described above. The * symbol denotes a difference with oxycodone used alone as compared to the combination of oxycodone with S norketameine and the +symbol denotes a difference from day 1. Data were analyzed with the SNK method, with P less than 0.05.

### Example 6

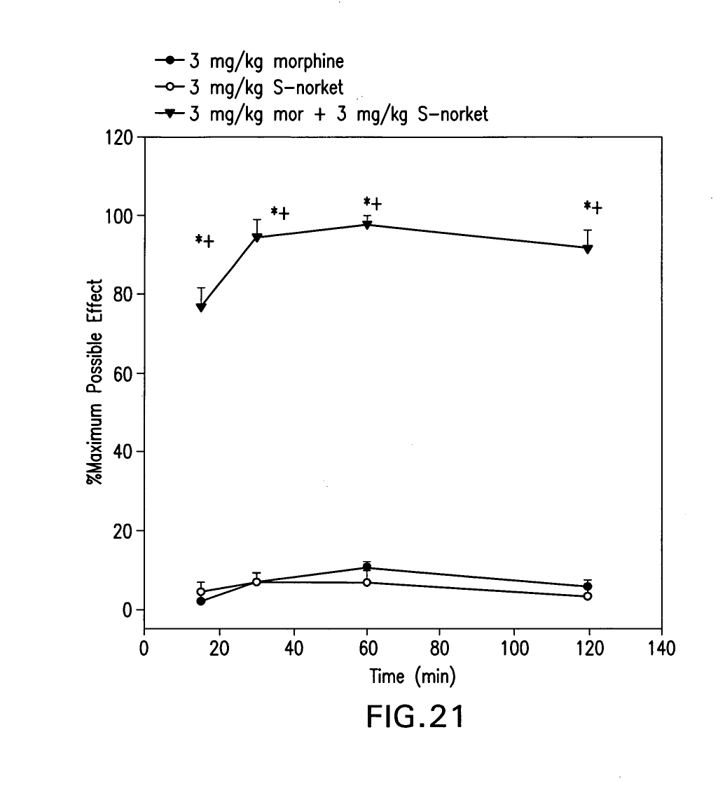

FIG. 21 depicts analgesic response of S-norketamine with morphine. A tail flick test as described above was administered to eight Sprague-Dawley rats as described above The * symbol denotes difference from morphine alone and the + symbol illustrates difference from S-norketamine alone as compared to the combination of S norketamine versus morphine. Data were analyzed with post-hoc SNK method, with P less than or equal to 0.001.

What is claimed is:

1. A method of alleviating pain in a subject in need thereof comprising administering to a subject in need thereof an effective amount of a first active ingredient and an effective amount of a second active ingredient,
the first active ingredient being selected from (S)-norketamine, (R)-norketamine, their respective salts, solvates, or prodrugs, or any combinations thereof,
the second active ingredient being selected from an opioid,
provided that the effective amount of the first active ingredient, if administered in the absence of the second active ingredient, would be insufficient to exert an optimal analgesic effect on the subject.

2. The method of claim 1 in which the effective amount of the second active ingredient, if administered in the absence of the first active ingredient, would be insufficient to exert an optimal analgesic effect on the subject.

3. The method of claim 1 in which the first and second active ingredients are administered separately.

4. The method of claim 1 in which the first and second active ingredients are administered together.

5. The method of claim 1 in which the second active ingredient is selected from fentanyl, sefentanil, alfentanil, morphine, hydromorphine, oxymorphine, methadone, oxycodone, hydrocodone, remifentanil, dihydrocodeine, ethylmorphine, nalbuphine, buprenorphine, dihydromorphine, normorphine, dihydroetorphine, butorphanol, pentazocine, phenazocine, codeine, meperidine, propoxyphene, tramadol, levorphanol, L-acetylmethadol, diacetylmorphine (heroin), etorphine, normethadone, noroxycodone, and norlevorphanol.

6. The method of claim 1 in which the second active ingredient is morphine.

7. A method of inhibiting tolerance to a narcotic analgesic in a subject in need thereof comprising co-administering to a subject in need thereof (S)-norketamine, (R)-norketamine, their respective salts, solvates, or prodrugs, or any combinations thereof with a narcotic analgesic,
the narcotic analgesic, if administered in the absence of the (S)-norketamine, (R)-norketamine, their respective salts, solvates, or prodrugs, or any combinations thereof, would induce in the subject a tolerance for the narcotic analgesic.

8. The method of claim 7 in which the narcotic analgesic would induce in the subject a tolerance for the narcotic analgesic after about one week of daily administration.

9. The method of claim 7 in which the narcotic analgesic is selected from fentanyl, sefentanil, alfentanil, morphine, hydromorphine, oxymorphine, methadone, oxycodone, hydrocodone, remifentanil, dihydrocodeine, ethylmorphine, nalbuphine, buprenorphine, dihydromorphine, normorphine, dihydroetorphine, butorphanol, pentazocine, phenazocine, codeine, meperidine, propoxyphene, tramadol, levorphanol, L-acetylmethadol, diacetylmorphine (heroin), etorphine, normethadone, noroxycodone, and norlevorphanol.

10. The method of claim 1 in which the narcotic analgesic is morphine.

11. The method of claim 7 in which the effective amount of the (S)-norketamine, (R)-norketamine, their respective salts, solvates, or prodrugs, or any combinations thereof would be insufficient to exert an optimal analgesic effect on the subject.

12. A method of alleviating pain in a subject in need thereof comprising administering to the subject a therapeutically effective amount of a first active ingredient and a therapeutically effective amount of a second active ingredient,
the first active ingredient being selected from (S)-norketamine, (R)-norketamine, their respective pharmaceutically acceptable salts, solvates, or prodrugs, or any combinations thereof,
the second active ingredient being selected from an opioid,
provided that (A) the therapeutically effective amount of the first active ingredient, if administered in the absence of the second active ingredient, would be insufficient to exert an optimal analgesic effect on the subject, (B) the therapeutically effective amount of the second active ingredient, if administered in the absence of the first active ingredient, would be insufficient to exert an optimal analgesic effect on the subject, or (C) the therapeutically effective amounts of both the first active ingredient and the second active ingredient, if each is administered alone, would be insufficient to exert an optimal analgesic effect on the subject.

13. The method of claim 12 in which the therapeutically effective amount of the second active ingredient, if administered in the absence of the first active ingredient, would be insufficient to exert an optimal analgesic effect on the subject.

14. The method of claim 12 in which the first and second active ingredients are administered separately.

15. The method of claim 12 in which the first and second active ingredients are administered together.

16. The method of claim 12 in which the second active ingredient is selected from fentanyl, sefentanil, alfentanil, morphine, hydromorphine, oxymorphine, methadone, oxycodone, hydrocodone, remifentanil, dihydrocodeine, ethylmorphine, nalbuphine, buprenorphine, dihydromorphine, normorphine, dihydroetorphine, butorphanol, pentazocine, phenazocine, codeine, meperidine, propoxyphene, tramadol, levorphanol, L-acetylmethadol, diacetylmorphine (heroin), etorphine, normethadone, noroxycodone, and norlevorphanol.

17. The method of claim 12 in which the second active ingredient is morphine or oxycodone.

18. A method of treating a neurodegenerative or neuropsychiatric disorder in a subject in need thereof comprising administering to the subject a therapeutically effective amount of a first active ingredient and a therapeutically effective amount of a second active ingredient,
the first active ingredient being selected from (S)-norketamine, (R)-norketamine, their respective pharmaceutically acceptable salts, solvates, or prodrugs, or any combinations thereof,
the second active ingredient being selected from an opioid,
provided that (A) the therapeutically effective amount of the first active ingredient, if administered in the absence of the second active ingredient, would be insufficient to exert an optimal analgesic effect on the subject, (B) the therapeutically effective amount of the second active ingredient, if administered in the absence of the first active ingredient, would be insufficient to exert an optimal analgesic effect on the subject, or (C) the therapeutically effective amounts of both the first active ingredient and the second active ingredient, if each is administered alone, would be insufficient to exert an optimal analgesic effect on the subject.

19. The method of claim 18 in which the disorder is Alzheimer's disease, Parkinson's syndrome or neurodegeneration.
